(19) United States
(12) Patent Application Publication
(10) Pub. No.: US 2023/0132217 A1
(43) Pub. Date: Apr. 27, 2023

(54) SPEECH RECOGNITION FOR HEALTHCARE COMMUNICATIONS

(71) Applicant: Hill-Rom Services, Inc., Batesville, IN (US)

(72) Inventors: John S. Schroder, Apex, NC (US); Eric D. Agdeppa, Cary, NC (US); Frederick Collin Davidson, Apex, NC (US); Catherine Infantolino, Neptune Beach, FL (US); Timothy J. Receveur, Apex, NC (US); Richard Joseph Schuman, Cary, NC (US); Dan R. Tallent, Hope, IN (US)

(21) Appl. No.: 18/046,607

(22) Filed: Oct. 14, 2022

Related U.S. Application Data
(60) Provisional application No. 63/263,047, filed on Oct. 26, 2021.

(51) Int. Cl. G16H 80/00; G16H 40/20; G16H 10/20; G10L 25/63; G10L 15/26; G10L 15/22; G06F 40/40

(52) U.S. Cl. CPC G16H 80/00; G16H 40/20; G16H 10/20; G10L 25/63; G10L 15/26; G10L 15/22; G06F 40/40; G10L 2015/223

(57) ABSTRACT

A communications system for a healthcare facility receives speech from a patient, and converts the speech to text to determine a patient request. The system processes the speech to determine an emotion classifier, and generates a message containing the patient request and the emotion classifier. The system identifies a caregiver based on the patient request, and sends the message to the caregiver.

### BACKGROUND

Patients in healthcare facilities generally require assistance. Nurse call buttons are available, but are not specific regarding a patient's request for assistance. For example, activating a nurse call button does not specify the patient's needs, emotional state, or condition.

Often, a caregiver must enter the patient's room to inquire about the patient's request for assistance. Assistance to the patient would be more efficient if the caregiver was familiar with the patient's needs prior to entering the patient's room.

### SUMMARY

In general terms, the present disclosure relates to speech recognition for healthcare communications. Various aspects are described in this disclosure, which include, but are not limited to, the following aspects.

In one aspect, a communications system for a healthcare facility comprises: at least one processing device; and a memory device storing instructions which, when executed by the at least one processing device, cause the at least one processing device to receive speech from a patient; convert the speech to text to determine a patient request; process the speech to determine an emotion classifier; generate a message containing the patient request and the emotion classifier; identify a caregiver based on the patient request; and send the message to the caregiver.

In another aspect, a method of healthcare based communications comprises receiving speech from a patient; converting the speech to text to determine a patient request; processing the speech to determine an emotion classifier; generating a message containing the patient request and the emotion classifier; identifying a caregiver based on the patient request; and sending the message to the caregiver.

In another aspect, a non-transitory computer readable storage medium storing instructions, which when executed by at least one processing device, cause the at least one processing device to receive speech from a patient; convert the speech to text to determine a patient request; process the speech to determine an emotion classifier; generate a message containing the patient request and the emotion classifier; identify a caregiver based on the patient request; and send the message to the caregiver.

### DESCRIPTION OF THE FIGURES

The following drawing figures, which form a part of this application, are illustrative of the described technology and are not meant to limit the scope of the disclosure in any manner.

FIG. 1 illustrates an example healthcare facility including a communications system having a patient bed, one or more mobile devices, and a nurse call server having a communications application installed thereon.

FIG. 2 schematically illustrates an example of the communications application installed on the nurse call server of FIG. 1.

FIG. 3 illustrates an example of a microphone and speaker unit on the patient bed that is part of the communications system of FIG. 1.

FIG. 4 illustrates an example of a method of healthcare based communications that can be performed by the communications system of FIG. 1.

FIG. 5 illustrates another example of a method of healthcare based communications that can be performed by the communications system of FIG. 1.

FIG. 6 illustrates an example of additional operations in the method of FIG. 5.

FIG. 7 illustrates an example of a user interface displayed on a mobile device of a caregiver of the communications system of FIG. 1.

FIG. 8 illustrates an example of a method that can be performed by the communications system of FIG. 1.

FIG. 9 illustrates another example of a user interface that includes a patient emotion summary generated in accordance with the operations of the method of FIG. 8.

FIG. 10 illustrates another example of a method that can be performed by the communications system of FIG. 1.

FIG. 11 illustrates another example of a method that can be performed by the communications system of FIG. 1.

FIG. 12 illustrates an example of a method of performing a clinical assessment that can be performed by the communications system of FIG. 1.

FIG. 13 schematically illustrates an example of a mobile device of the communications system of FIG. 1.

FIG. 14 illustrates an example of a user interface displayed on a status board.

FIG. 15 illustrates an example of an alarming algorithm that uses an emotion classifier of a patient as a factor in determining whether to trigger an alarm.

### DETAILED DESCRIPTION

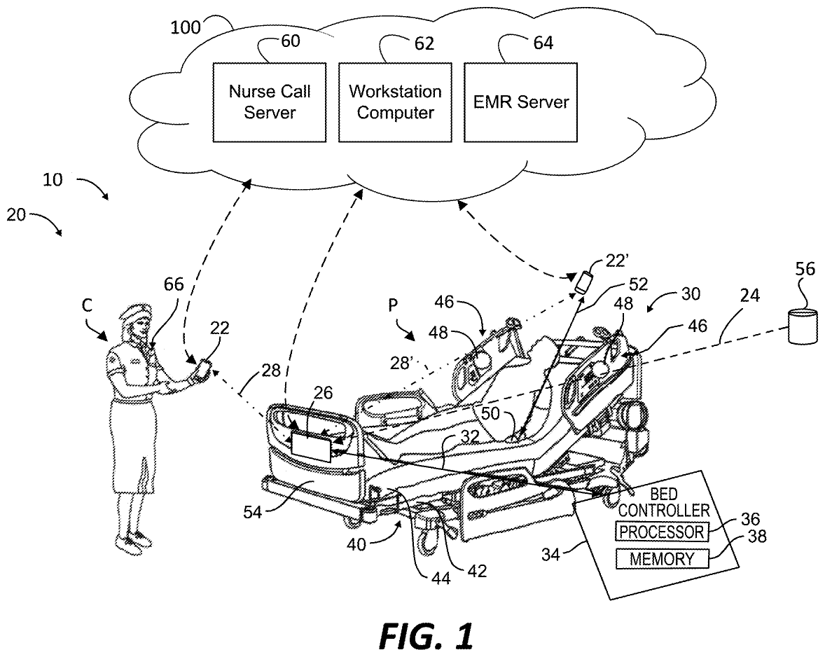

FIG. 1 illustrates an example of a communications system 20 in a healthcare facility 10. The communications system 20 facilitates communication between a patient P and a caregiver C. As will be described in more detail, the communications system 20 provides context to communications received by the caregiver C from the patient P. For example, the communications system 20 can utilize speech recognition to determine a patient request and an emotional state based on captured audio and speech inputs of the patient P, and can generate a message that includes the patient request and emotional state for receipt by the caregiver C. Additionally, the communications system 20 can utilize speech recognition and natural language processing to guide the patient P through a clinical assessment initiated by the caregiver C.

As shown in FIG. 1, the patient P is supported on a patient bed 30, and the caregiver C carries a mobile device 22 which receive the messages from the patient P, that include a patient request for help or assistance and an emotional state of the patient. The messages can be generated from a microphone and speaker unit 48 provided on the patient bed 30, or from a mobile device 22′ that is operated by the patient P while supported on the patient bed 30. In some instances, the messages are nurse calls, or similar types of healthcare communications.

In FIG. 1, the mobile device 22 communicates with a wireless access module (WAM) 26 of patient bed 30, as shown by wireless link 28. The WAM 26 can send messages to the mobile device 22 of the caregiver C through the wireless link 28, and the caregiver C can use the mobile device 22 to send messages through the wireless link 28 to the WAM 26. Accordingly, the wireless link 28 between mobile device 22 and the WAM 26 is bidirectional.

The mobile device 22′ also communicates with the WAM 26, as indicated by wireless link 28′. The mobile device 22′ can also communicate with the mobile device 22 of the caregiver C through a network 100 without having to pass through the WAM 26. The network 100 can include any type of wired or wireless connections or any combinations thereof. Examples of wireless connections include broadband cellular network connections such as 4G or 5G. In some instances, wireless connections can also be accomplished using Bluetooth, Wi-Fi, and the like.

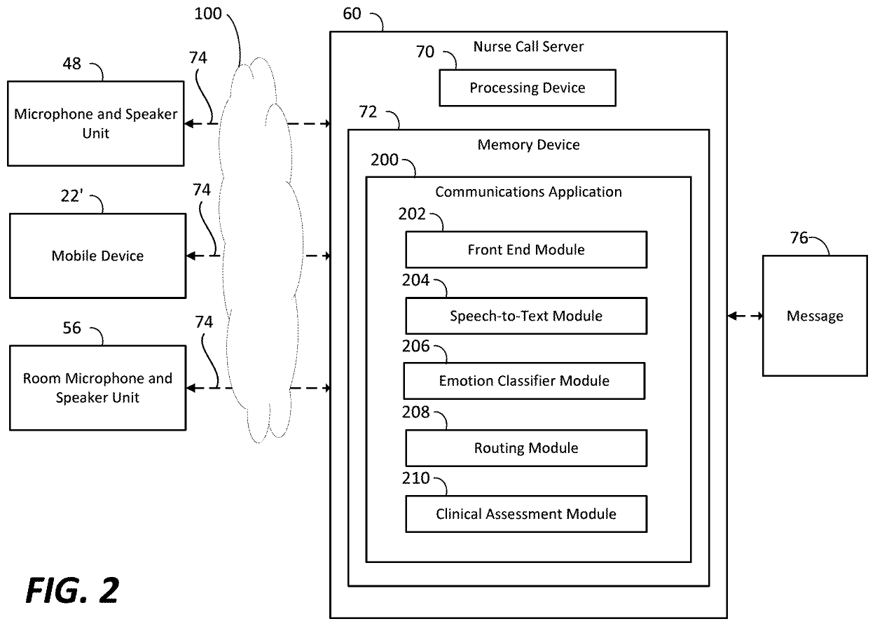

As shown in FIG. 1, a nurse call server 60 is included within the network 100. The nurse call server 60 manages communications between the patient bed 30, mobile device 22 of the caregiver C, mobile device 22′ of the patient P, and a workstation computer 62. In one embodiment, the nurse call server 60 includes a communications application 200, shown in FIG. 2, which generates the messages that include the patient request and emotional state of the patient P, and which are received by the mobile device 22 of the caregiver C.

As further shown in FIG. 1, the workstation computer 62 is also included within the network 100. The workstation computer 62 can be located in a nurses' station, where the caregiver C and other caregivers and staff of the healthcare facility 10 work when not working directly with the patient P, such as where they can perform administrative tasks.

The mobile devices 22, 22′ of the caregiver C and patient P, respectively, can include smartphones, tablet computers, or similar types of portable computing devices. In one embodiment, the mobile device 22′ includes the communications application 200, shown in FIG. 2, which generates the messages that include the patient request and emotional state of the patient P, and which are received by the mobile device 22 of the caregiver C.

The WAM 26 is communicatively connected to a bed controller 34 via a wired or wireless link 32. The bed controller 34 includes at least one processing device 36, such as one or more microprocessors or microcontrollers that execute software instructions stored on a memory device 38 to perform the functions and operations described herein. In one embodiment, the memory device 38 of the bed controller 34 stores the communications application 200, shown in FIG. 2, which generates the messages that include the patient request and emotional state of the patient P, and which are received by the mobile device 22 of the caregiver C.

The bed controller 34 can include circuit boards, electronics modules, and the like that are electrically and communicatively interconnected. The bed controller 34 can further include circuitry, such as a processor, a microcontroller, a field programmable gate array (FPGA), an application specific integrated circuit (ASIC), reconfigurable circuitry, System on Chip (SoC), Programmable System on Chip (PSoC), Computer on Module (CoM), and System on Module (SoM), and the like to perform the functions and operations described herein.

While the WAM 26 is shown in the example of FIG. 1 as being coupled to a footboard 54 of the patient bed 30, it is possible for the WAM 26 to be carried by or otherwise be included on other portions of the patient bed 30. Also, the WAM 26 and bed controller 34 may be included on the same printed circuit board. Accordingly, in at least some embodiments, the WAM 26 may be implemented as part of the bed controller 34.

Still referring to FIG. 1, the patient bed 30 has a frame 40 including a support deck 42 supporting a mattress 44. A head end siderail 46 is coupled to each side of a head section of the support deck 42 such that patient bed 30 has two head end siderails. The microphone and speaker unit 48 is provided on at least one of the head end siderails 46. In the example shown in FIG. 1, a microphone and speaker unit 48 is provided on each head end siderail 46.

The microphone and speaker units 48 are capable of detecting audio and receiving speech inputs from the patient P. Each of the microphone and speaker units 48 can be provided as a single unit. Alternatively, each microphone and speaker unit 48 can include separate microphone and speaker components that are part of the circuitry of the head end siderail 46.

Audio and speech inputs from the patient P can be captured by one or more of the microphone and speaker units 48 provided on one or more of the head end siderails 46. In one embodiment, the microphone and speaker units 48 communicate the audio and speech inputs to the WAM 26, which transmits the audio and speech inputs to the nurse call server 60 via the network 100, and the nurse call server 60 uses speech recognition to convert the audio and speech inputs into the messages that include the patient request and emotional state for wireless transmission via the network 100 to the mobile device 22 of the caregiver C.

In another embodiment, the microphone and speaker units 48 communicate the audio and speech inputs to the bed controller 34, and the bed controller 34 uses speech recognition to convert the audio and speech inputs into the messages that include the patient request and emotional state. Thereafter, the messages can be wirelessly transmitted by the WAM 26 to the mobile device 22 of the caregiver C via the wireless link 28 or using the network 100.

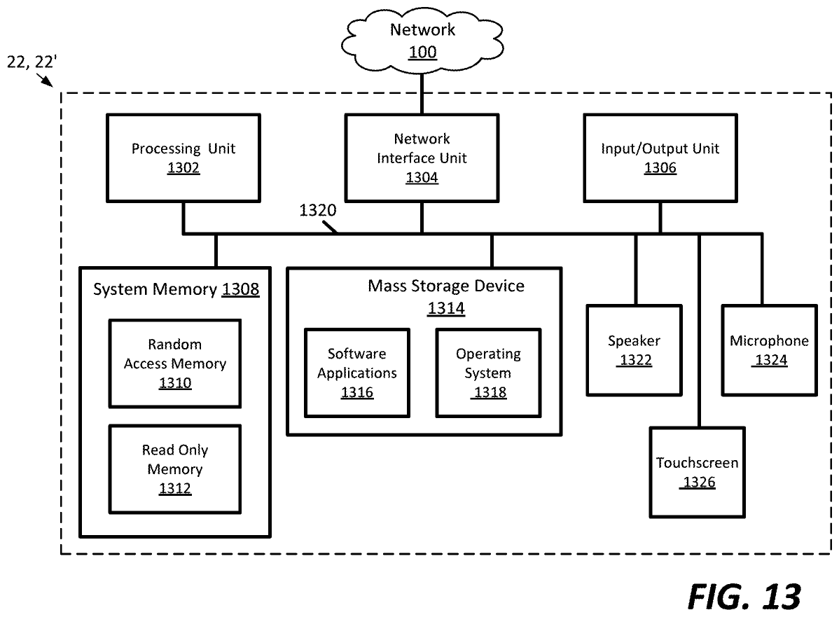

The mobile devices 22, 22′ each include a speaker 1322 and a microphone 1324, shown in FIG. 13, that can be used in addition to, or as an alternative to, the microphone and speaker units 48 to record audio and enter speech inputs of the patient P. The mobile device 22′ can send the recorded audio and speech inputs from the microphone 1324 to the nurse call server 60 via the network 100, or to the WAM 26 via the wireless link 28′. Additionally, messages, audio, speech inputs, and the like can be sent from the patient bed 30 to the mobile device 22′ via the wireless link 28′ connecting the WAM 26 to the mobile device 22′.

In the example illustrated in FIG. 1, a wrist band 50 is worn by the patient P and provides a wireless signal 52 to the mobile device 22′. The wireless signal 52 from the wrist band 50 can include a patient ID (e.g., patient's medical record number (MRN), a randomly assigned number, the patient's room number and name, and the like). The mobile device 22′ can include the patient ID with the audio and speech inputs that are sent to the WAM 26 via the wireless link 28′, and in the messages sent to the mobile device 22 of the caregiver C. The patient ID can be used to identify or associate the message as belonging to the patient P.

In one embodiment, the mobile device 22′ of the patient P uses speech recognition to convert the audio and speech inputs from the patient P into the messages that include the patient request and emotional state. Thereafter, the mobile device 22′ of the patient P can communicate the messages to the mobile device 22 of the caregiver C through the network 100.

In another embodiment, the mobile device 22′ transmits the audio and speech inputs from the patient P to the nurse call server 60 via the network 100, and the nurse call server 60 uses speech recognition to convert the audio and speech inputs into the messages that include the patient request and emotional state. Thereafter, the nurse call server 60 can wirelessly transmit the messages to the mobile device 22 of the caregiver C using the network 100.

In another embodiment, the mobile device 22′ communicates the audio and speech inputs from the patient P to the WAM 26 via wireless link 28′, and the bed controller 34 receives the audio and speech inputs from the WAM 26. Thereafter, the bed controller 34 uses speech recognition to convert the audio and speech inputs into the messages that include the patient request and emotional state, which can be wirelessly transmitted by the WAM 26 to the mobile device 22 of the caregiver C using the wireless link 28 or the network 100.

As further shown in FIG. 1, the communications system 20 can include a room microphone and speaker unit 56 that detects audio and speech inputs from the patient P. The room microphone and speaker unit 56 can be mounted to a wall, ceiling, fixture, furniture, or equipment in an area where the patient P is located. For example, the room microphone and speaker unit 56 can be placed on a nightstand adjacent to the patient bed 30, mounted to a room wall or a ceiling adjacent to the patient bed 30, or can be mounted to the patient bed 30 itself.

The room microphone and speaker unit 56 is communicatively connected to the WAM 26 through a wired or wireless link 24, and as noted above, the bed controller 34 is communicatively connected to the WAM 26 via the wired or wireless link 32. Thus, the room microphone and speaker unit 56 can capture audio and speech inputs, and send the audio and speech inputs to the WAM 26 for processing by the bed controller 34. In some embodiments, the room microphone and speaker unit 56, the microphone and speaker units 48, and the mobile device 22′ cooperate with each other to provide the communications system 20 with an array of microphones that can detect audio and receive speech inputs from the patient P.

In one embodiment, the audio and speech inputs are communicated from the room microphone and speaker unit 56 to the WAM 26 via the wired or wireless link 24, and the WAM 26 transmits the audio and speech inputs to the nurse call server 60 via the network 100. Thereafter, the nurse call server 60 uses speech recognition to convert the audio and speech inputs into the messages that include the patient request and emotional state for wireless transmission to the mobile device 22 of the caregiver C using the network 100.

In another embodiment, the audio and speech inputs are communicated from the room microphone and speaker unit 56 to the WAM 26 via the wired or wireless link 24, and the bed controller 34 receives the audio and speech inputs from the WAM 26. The bed controller 34 then uses speech recognition to convert the audio and speech inputs into the messages that include the patient request and emotional state for wireless transmission by the WAM 26 to the mobile device 22 of the caregiver C using the wireless link 28 or the network 100.

As shown in FIG. 1, the WAM 26 is communicatively connected to the nurse call server 60 via the network 100, which is positioned off of the patient bed 30. The nurse call server 60 can be located on-premises in the healthcare facility 10, can be remotely located outside of the healthcare facility 10, or can be a cloud server. The WAM 26 can also be connected to additional systems and devices in the healthcare facility 10, via the network 100.

In one embodiment, the nurse call server 60 provides continuous speech processing (CSP) recognition and natural language processing (NLP) services by using one or more software applications installed on a memory device 72 (see FIG. 2) of the nurse call server 60. In such embodiments, the audio and speech inputs recorded from the microphone and speaker unit 48, the mobile device 22′, or the room microphone and speaker unit 56 are transmitted to the nurse call server 60 via the network 100. The nurse call server 60 processes the audio and speech inputs to provide context to nurse calls that are sent to the mobile device 22 of the caregiver C.

In an alternative embodiment, the bed controller 34 provides the CSP recognition and NLP services by using one or more software applications installed on the memory device 38 of the bed controller 34. In such embodiments, the audio and speech inputs recorded from the microphone and speaker unit 48, mobile device 22′, or room microphone and speaker unit 56 are transmitted to the bed controller 34 via the WAM 26, and the bed controller 34 processes the audio and speech inputs to provide context to nurse calls sent from the patient bed 30 or mobile device 22′ of the patient P to the mobile device 22 of the caregiver C.

In yet another embodiment, the mobile device 22′ of the patient P provides the CSP recognition and NLP services by using one or more software applications installed on a memory device of the mobile device 22′. In such embodiments, the audio and speech inputs recorded by the microphone of the mobile device 22′ are processed by the mobile device 22′ to provide context to nurse calls sent from the mobile device 22′ to the mobile device 22 of the caregiver C.

As further shown in FIG. 1, an electronic medical record (EMR) server 64 can be included within the network 100. The EMR server 64 stores data collected from the patient P in an electronic medical record (EMR) (alternatively termed electronic health record (EHR)) of the patient P. Examples of data that can be collected from the patient P include patient vital signs and other physiological variables including patient weight and warning scores.

FIG. 2 schematically illustrates an example of the communications application 200 installed on a memory device 72 of the nurse call server 60. As described above, the communications application 200 can also be installed on the memory device 38 of the bed controller 34, and/or on memory devices of the mobile devices 22, 22′. As shown in FIG. 2, nurse call server 60 includes a processing device 70, which is an example of a processing unit such as a central processing unit (CPU). The processing device 70 can include one or more central processing units (CPU). In some examples, the processing device 70 can include one or more digital signal processors, field-programmable gate arrays, or other electronic circuits.

The memory device 72 operates to store data and instructions, including the communications application 200, for execution by the processing device 70. The memory device 72 includes computer-readable media, which may include any media that can be accessed by the nurse call server 60. By way of example, computer-readable media include computer readable storage media and computer readable communication media.

Computer readable storage media includes volatile and nonvolatile, removable and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the nurse call server 60. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

As shown in FIG. 2, the microphone and speaker unit 48, mobile device 22′, and room microphone and speaker unit 56 are connected to the nurse call server 60 via the network 100. The microphone and speaker unit 48, mobile device 22′, and room microphone and speaker unit 56 transfer audio and speech inputs 74 to the nurse call server 60 via the network 100.

As further shown in FIG. 2, the communications application 200 includes several modules or sub-applications for processing the recorded audio and speech inputs 74. For example, the communications application 200 includes a front end module 202, a speech-to-text module 204, an emotion classifier module 206, a routing module 208, and a clinical assessment module 210. The communications application 200 processes the recorded audio and speech inputs 74 to generate one or more outputs, such as a message 23 that can be delivered to the mobile device 22 of the caregiver C to assist with a patient request.

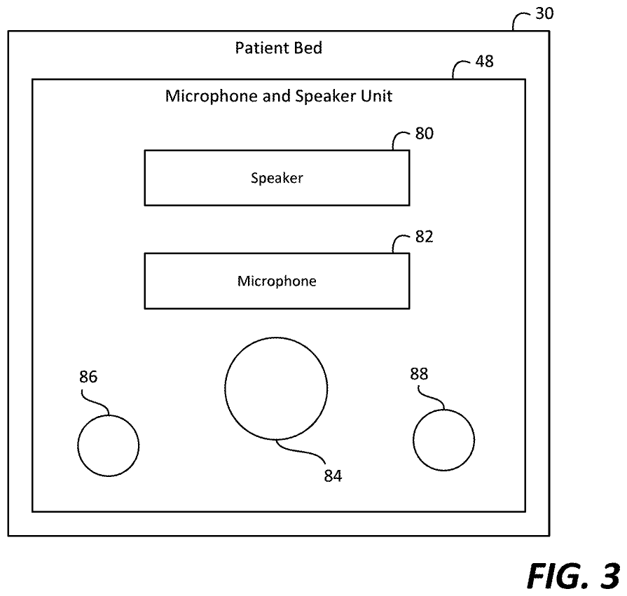

FIG. 3 illustrates an example of the microphone and speaker unit 48 installed on the patient bed 30. As will be described in more detail, the microphone and speaker unit 48 can be used to summarize a reason for submitting a nurse call. This allows a recipient of the nurse call (e.g., the caregiver C) to understand the reason for the nurse call without having to first enter the room where the patient P is located and without much additional effort from the patient P.

As shown in FIG. 3, the microphone and speaker unit 48 includes a speaker 80 and a microphone 82. In this example, the speaker 80 and microphone 82 are separate components. Alternatively, in other examples, the speaker 80 and microphone 82 can be combined into a single component or unit that operates as both a speaker and a microphone.

The microphone and speaker unit 48 further includes a nurse call button 84 that the patient P can press to submit a nurse call, and first and second indicators 86, 88. In some examples, the first and second indicators 86, 88 are light-emitting diodes (LEDs). The first indicator 86 emits a light to indicate that the nurse call has been submitted and is active, and the second indicator 88 emits a light to indicate that the microphone 82 is recording the audio or speech input of the patient P. As will be described in more detail with reference to the method 400, operation of the microphone and speaker unit 48 by the patient P is intuitive.

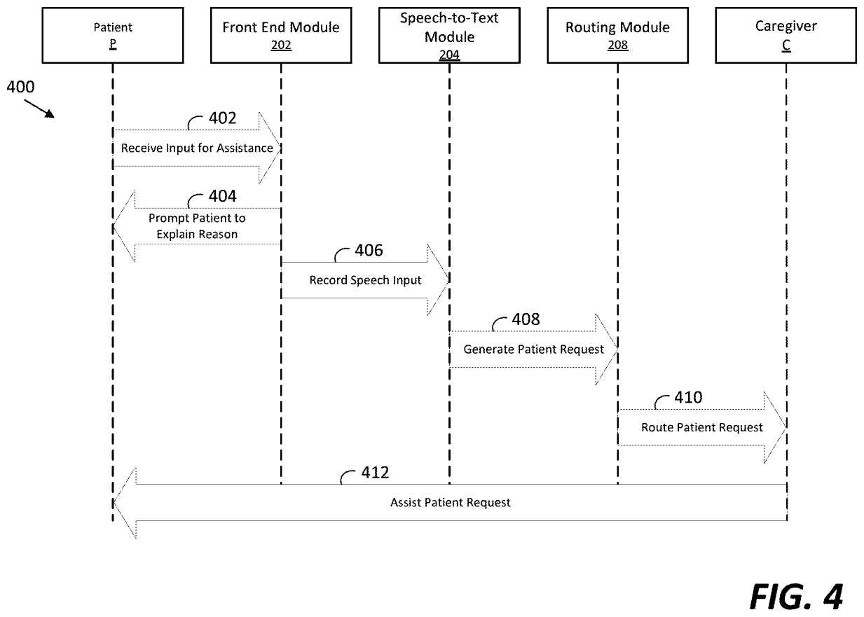

FIG. 4 illustrates an example of a method 400 of healthcare based communications that can be performed by the communications system 20. While the following description describes the method 400 with reference to the microphone and speaker unit 48 on the patient bed 30, the method 400 can also be performed through the mobile device 22′.

Referring now to FIGS. 1-4, the method 400 includes an operation 402 of receiving an input from the patient P requesting assistance. The input received in operation 402 can be detected from the patient P pressing the nurse call button 84. The operation 402 of receiving the input from the nurse call button 84 can be performed by the front end module 202.

Next, the method 400 includes an operation 404 of prompting the patient P to explain the reason for the assistance. The prompt is generated through the speaker 80 of the microphone and speaker unit 48. The prompt can include a phrase such as “Please summarize your reason for the call”, or something similar. The operation 404 of prompting the patient P to explain the reason for the assistance can be performed by the front end module 202.

Next, the method 400 includes an operation 406 of recording a speech input from the patient P in response to the prompt from operation 404. The speech input can be recorded by the microphone 82 of the microphone and speaker unit 48. During operation 406, the front end module 202 can illuminate the second indicator 88 on the microphone and speaker unit 48 to indicate that the microphone 82 is operating to record the audio or speech input of the patient P.

Next, the method 400 includes an operation 408 of generating a patient request by converting the speech input recorded in operation 406 to text. Operation 408 is performed by the speech-to-text module 204 of the communications application 200. Operation 408 can include parsing and summarizing the text to generate the patient request. For example, the text can be condensed to a short summary or mapped to a predefined care category that is used for the patient request. As an illustrative example, the full text of the speech input can include “Nurse Smith, I would like to have some ice chips please”, which can be condensed to “ice chips”.

Next, the method 400 includes an operation 410 of routing the patient request to an appropriate caregiver. Operation 410 is performed by the routing module 208 of the communications application 200. Operation 410 can include identifying an appropriate caregiver by matching the patient request to one or more predefined roles of the caregivers. For example, when the text is condensed to a short summary or mapped to a predefined care category, the short summary or predefined care category can be mapped to the one or more predefined roles of the caregivers within the healthcare facility 10 to identify an appropriate caregiver.

In some examples, operation 410 includes routing the patient request to the mobile device 22 of a caregiver who is identified as being able to fulfill the patient request based on their role in the healthcare facility 10. Alternatively, or in addition, operation 410 can include routing the patient request to the workstation computer 62 to alert one or more caregivers about the patient request, and can include identification of certain caregivers who are identified as being able to fulfill the patient request based on their role in the healthcare facility 10.

Upon receiving the patient request, the caregiver C can assist fulfillment of the patient request, as indicated by operation 412. In some instances, operation 412 can include opening an audio link between the microphone and speaker unit 48 and the mobile device 22 of the caregiver C to allow for two-way audio communications between the patient P and the caregiver C.

In an alternative embodiment, the method 400 can be performed through the mobile device 22′ operated by the patient P. For example, instead of receiving an input from the nurse call button 84 on the microphone and speaker unit 48, operation 402 can include receiving an input from a graphical user interface displayed on a touchscreen 1326 (see FIG. 13) of the mobile device 22′. In certain instances, the input on the graphical user interface is a “voice request”. Thereafter, the operation 404 of prompting the patient P to explain the reason for the assistance is generated through the speaker 1322 (see FIG. 13) of the mobile device 22′, and the operation 406 of recording the speech input from the patient P is done through the microphone 1324 (see FIG. 13) of the mobile device 22′. Operations 408-412 can be performed similarly regardless of whether the microphone and speaker unit 48 or the mobile device 22′ are used.

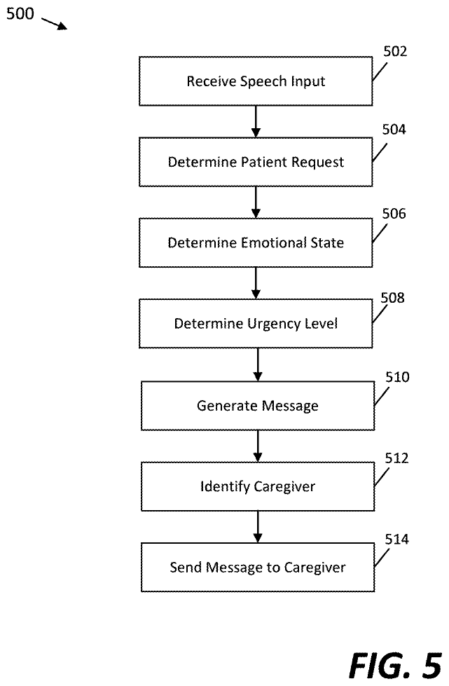

FIG. 5 illustrates an example of a method 500 of healthcare based communications that can be performed by the communications system 20. The method 500 can be used to add emotional content classifiers to add context to the healthcare based communications.

As shown in FIG. 5, the method 500 includes an operation 502 of receiving a speech input from the patient P. In accordance with the examples described above, the speech input can be received from the microphone 82 on the microphone and speaker unit 48, the microphone 1324 on the mobile device 22′, or from the room microphone and speaker unit 56.

Next, the method 500 includes an operation 504 of determining a patient request by converting the speech input recorded in operation 406 to text. Operation 504 can be performed by the speech-to-text module 204, as shown in FIG. 2. Operation 504 can be substantially similar to operation 408 described above with respect to the method 400. For example, operation 504 can include parsing and summarizing the text to determine the patient request such as by condensing the text to a short summary or mapping the text to a predefined care category.

Next, the method 500 includes an operation 506 of determining an emotional state from the speech input. The emotional state can be determined by looking at certain features in the speech input received from the patient P, such as the tone, pitch, jitter, energy, rate, length and number of pauses, and the like. Operation 506 can be performed by the emotion classifier module 206, which can utilize artificial intelligence to analyze the features of the audio and speech input received from the patient P to determine the emotional state. The emotion classifier module 206 can store a codex of emotional states and associated emotional content classifiers that can be determined from the speech input such as, without limitation, happy, excited, sad, depressed, scared, frustrated, angry, nervous, anxious, tired, relaxed, and bored emotional states.

Also, in some examples, the text from the conversion of the speech input performed in operation 504 can be used to determine the emotional state. For example, certain words in the converted text can be associated with an emotional state such as words that correlate to happiness, enthusiasm, anger, sadness, anxiety, or boredom. Thus, the text of the converted audio and speech inputs may also be used to determine the emotional state of the patient P.

In some embodiments, the method 500 includes an operation 508 of determining an urgency level from the speech input. The urgency level can correspond to the emotional state determined in operation 506. For example, higher urgency levels can be associated with angry or scared emotional states, whereas lower urgency levels can be associated with happy or bored emotional states. As an illustrative example, the phrase “help me” has a higher urgency level when the emotional state is “scared” than when the emotional state is “bored”.

Additionally, the urgency level can be determined using the same features that are used for determining the emotional state. For example, the urgency level can be determined from the tone, pitch, jitter, energy, rate, length and number of pauses in the speech input, as well as from the words identified in the converted text of the speech input.

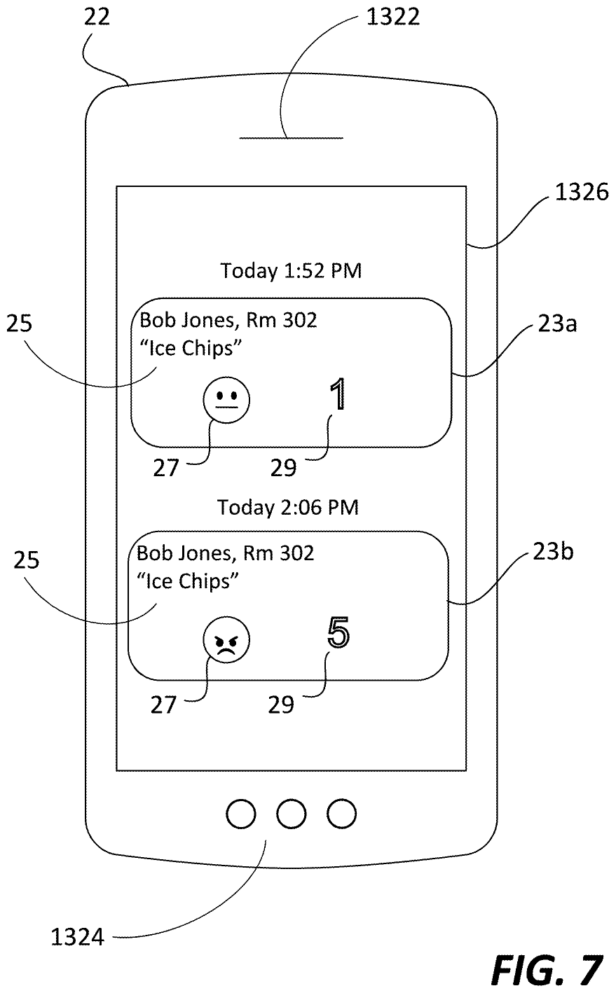

Next, the method 500 includes an operation 510 of generating a message that includes the patient request determined from operation 504, the emotional state determined from operation 506, and the urgency level determined from operation 508. Examples of the messages generated in operation 510 are shown in FIG. 7, which will be described in more detail below.

Referring back to FIG. 5, the method 500 next includes an operation 512 of identifying a caregiver based on the patient request determined from operation 504. For example, operation 512 can include identifying an appropriate caregiver in the healthcare facility 10 by matching the patient request to one or more predefined roles in the healthcare facility 10.

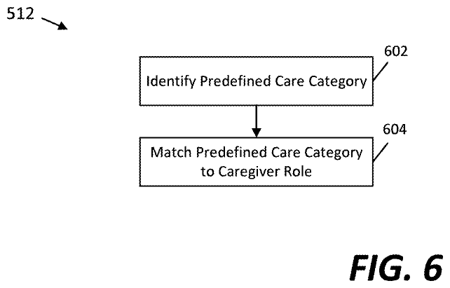

FIG. 6 illustrates an example of the operation 512 in further detail. As shown in FIG. 6, operation 512 can include a step 602 of identifying a predefined care category based on the patient request. For example, step 602 can include parsing, summarizing, and/or condensing the patient request to a predefined care category such as medication administration, medical equipment operation, counseling, bathroom assistance, linens request, and the like.

Next, operation 512 can include a step 604 of matching the predefined care category determined from step 602 to a role of a caregiver in the healthcare facility 10. For example, the caregivers may have roles that authorize them to perform certain tasks such as to assist the patient P to use the bathroom or to bring fresh linens, but that do not authorize them to perform other tasks such as to provide counseling on a treatment plan or operate medical equipment.

Also, operation 512 can include identifying an appropriate caregiver based on the emotional state of the patient P determined from operation 506. For example, when the emotional state is depressed or even suicidal, operation 512 can identify a caregiver who is mental health expert (e.g., psychologist), and is thus better able to assist the patient P.

Also, operation 512 can include identifying a caregiver based on the location where the patient P is admitted in the healthcare facility 10, and the assigned location of the caregiver. For example, when the patient P is admitted to a room number or unit in the healthcare facility, operation 512 can include identifying caregivers assigned to that room number or unit.

Referring back to FIG. 5, the method 500 next includes an operation 514 of sending the message generated in operation 510 to the caregiver identified in operation 512. In some examples, operation 514 includes routing the message generated in operation 510 to the mobile device 22 of a caregiver identified in operation 512. Alternatively, or in addition, operation 514 can include routing the message generated in operation 510 to the workstation computer 62 where the caregiver identified in operation 512 is located within the healthcare facility 10.

FIG. 7 illustrates an example of first and second messages 23a, 23b that can be displayed on the mobile device 22 of the caregiver C in accordance with the operations of the method 500, as described above. As shown in FIG. 7, the first message 23a includes a patient request 25 “Ice Chips” which can be summarized or condensed from a speech input from the patient P such as “Hi, can I please have some ice chips?”, an emotion classifier 27 indicating a neutral emotional state, and an urgency level of 1 indicating a low urgency level.

In contrast, the second message 23b, which is received after the first message 23a, includes the patient request 25 “Ice Chips” which can be summarized or condensed from a speech input from the patient P such as “Hey! Where's my ice chips!?”, an emotion classifier 27 indicating a frustrated or angry emotional state, and an urgency level of 5 indicating a higher urgency level. Thus, the emotional content of the textual patient request can be inferred from the emotion classifiers 27, when the patient request 25 “Ice Chips” is the same in both the first and second messages 23a, 23b due to the patient request being condensed to a short summary or mapped to a predefined care category. Advantageously, this allows the caregiver C to be aware of the patient's emotional state when the caregiver C receives the patient request on their mobile device 22 as a short summary or a predefined care category, which can improve the care provided by the caregiver C to the patient B, and thereby improve the patient P's satisfaction.

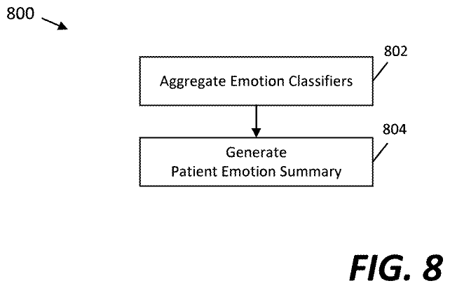

FIG. 8 illustrates an example of a method 800 that can be performed by the communications system 20. As shown in FIG. 8, the method 800 can include an operation 802 of aggregating the emotion classifiers 27 sent in the messages 23 from the patient P to the caregivers C during the patient P's admission in the healthcare facility 10. For example, operation 802 can include aggregating the number of happy emotion classifiers, aggregating the number of frustrated or angry emotion classifiers, and so on.

Next, the method 800 includes an operation 804 of generating a patient emotion summary based on the aggregated emotion classifiers determined from operation 802. The patient emotion summary can help caregivers better understand the patient P's emotional state over a period of time such as during a particular day or days, or the overall emotional state of the patient P during their admission in the healthcare facility 10. The patient emotion summary can help improve the care provided by the caregivers to the patient P, and can also be used as a metric for patient satisfaction to improve Hospital Consumer Assessment of Healthcare Providers and Systems (HCAHPS) scores in the healthcare facility 10.

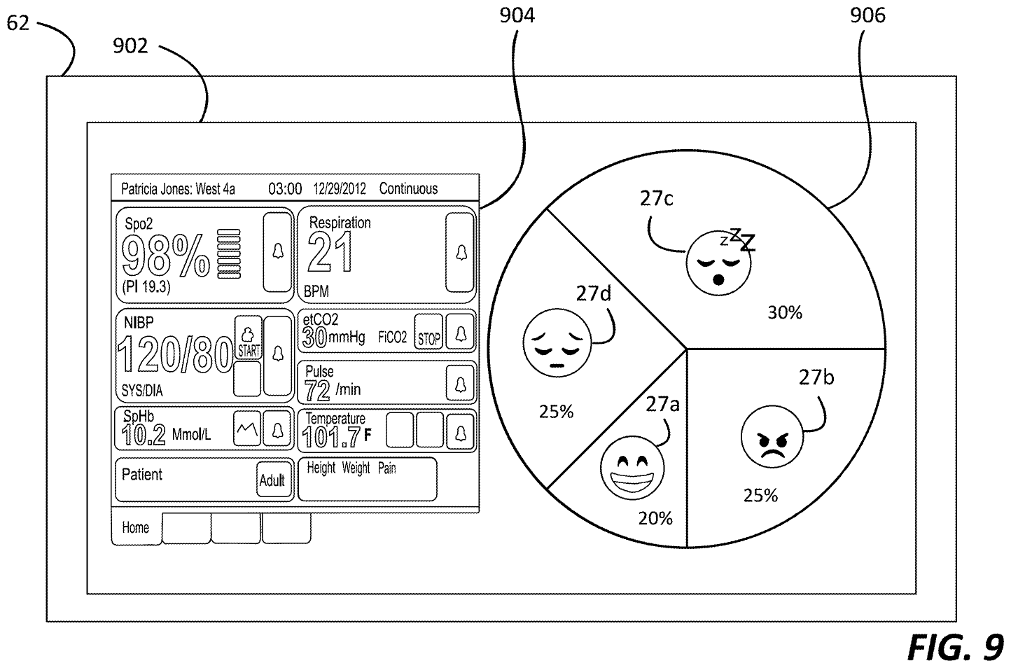

FIG. 9 illustrates an example of a user interface 902 that includes a patient emotional state summary 906 that is generated in accordance with the operations of the method 800. In the example of FIG. 9, the user interface 902 is displayed on the workstation computer 62. In further examples, the user interface 902 can be displayed on the mobile device 22 of the caregiver C such as on a mobile phone or tablet computer, on a bedside monitor positioned next to the patient bed 30 such as a spot monitor, or on the patient bed 30. In further examples, the user interface 902 can be displayed on a status board display for a floor or unit within the healthcare facility 10.

As shown in FIG. 9, the user interface 902 can include patient data 904 that can be acquired from the EMR of the patient P stored on the EMR server 64. The patient data 904 can include bibliographical information of the patient P such as the patient P's name, room number, date of admission, and the like, and can also include vital signs and other physiological variables collected from the patient P such as heart rate, respiration rate, SpO2, etCO2, non-invasive blood pressure (NIBP), temperature, pain level, early warning score, and the like.

The patient emotional state summary 906 can be displayed next to the patient data 904 as a chart that illustrates the emotional states of the patient P determined from the audio and speech inputs received from the patient P over a period of time, such as during a particular day or week, or overall, during the patient P's admission in the healthcare facility 10. Also, in addition to displaying the patient emotional state summary 906, the patient emotional state summary 906 can also be stored to the electronic medical record of the patient P stored in the EMR server 64.

Each emotional state in the patient emotional state summary 906 includes an emotion classifier 27 that identifies the emotional state and a percentage that indicates a relative amount that the patient P experienced the emotional state over a period of time. In the example shown in FIG. 9, the patient emotional state summary 906 includes an emotion classifier 27a indicating that the patient P experienced a happy emotional state during 20% of the time, an emotion classifier 27b indicating that the patient P experienced a frustrated or angry emotional state during 25% of the time, an emotion classifier 27c indicating that the patient P experienced a tired emotional state during 30% of the time, and an emotion classifier 27d indicating that the patient P experienced a sad emotional state during 25% of the time. The patient emotional state summary 906 can include additional types of emotion classifiers 27 stored in the codex of emotional states, depending on the emotional states of the patient P determined from the audio and speech inputs received by the communications system 20 over the period of time.

The patient emotional state summary 906 is updated in real-time based on the audio and speech inputs from the patient P. The patient emotional state summary 906 allows caregivers and staff in the healthcare facility 10 to improve the emotional state of the patient P, such as by increasing the happy emotional state identified by the emotion classifier 27a, and decreasing the frustrated and sad emotional states identified by the emotion classifiers 27b, 27d.

As described above, in some example embodiments, the patient emotional state summary 906 can be displayed on a status board display for a floor or unit within the healthcare facility 10 that allows caregivers and staff of the healthcare facility 10 to view of how patient P's stay in the healthcare facility 10 is going relative to other patients. The patient emotional state summary 906 can be used by an office of patient experience, which tracks patient satisfaction across the healthcare facility 10 and handles issues with dissatisfied patients.

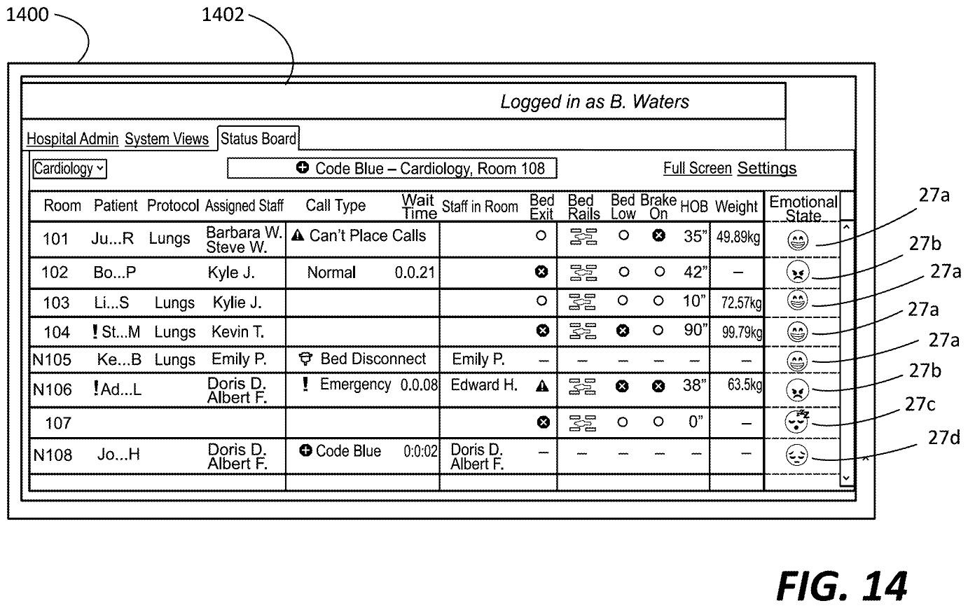

FIG. 14 illustrates an example of a user interface 1402 displayed on a status board 1400. In the example provided in FIG. 14, the user interface 1402 includes an emotion classifier 27 for each patient listed on the status board 1400 along with other information such as room number, patient name, assigned staff, call type, wait time, and the like. The emotion classifier 27 can represent a current emotion state of the patient, or the most common or prevalent emotion state during the patient's stay in the healthcare facility 10.

In further example embodiments, emotion classifiers 27 that represent the current emotion state of the patient, or the most common or prevalent emotion state during the patient's stay in the healthcare facility 10, can be used in one or more alarming algorithms.

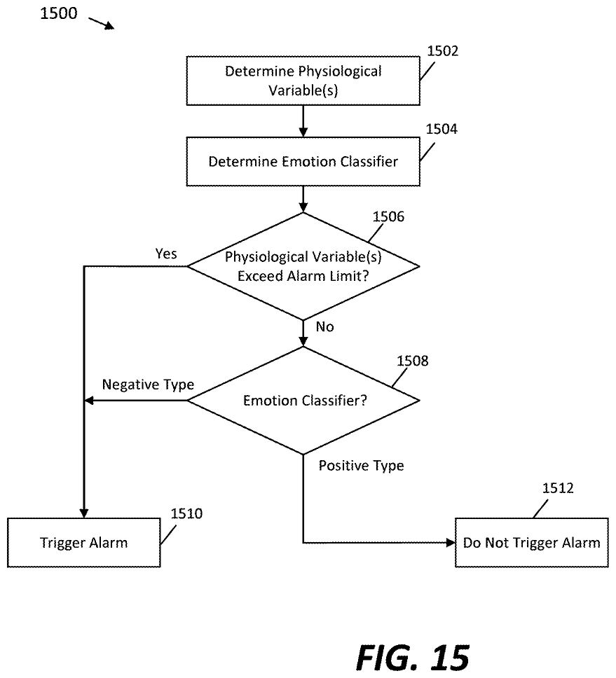

FIG. 15 illustrates an example of an alarming algorithm 1500 that uses an emotion classifier 27 of a patient as a factor in determining whether to trigger an alarm. The alarming algorithm 1500 includes an operation 1502 of determining one or more physiological variable of a patient such as heart rate, respiration rate, SpO2, etCO2, non-invasive blood pressure (NIBP), temperature, pain level, early warning score, and the like.

Next, the alarming algorithm 1500 includes an operation 1504 of determining an emotion classifier 27 the patient in accordance with the examples described above. In some examples, operations 1502, 1504 can occur simultaneously such that the physiological variables and emotion classifiers are determined at the same time. Operation 1504 can also include classifying the emotion classifier as a positive type of emotion classifier that indicates good mental health, or as a negative type of emotion classifier that indicates poor mental health. Illustrative examples of a positive type of emotion classifier can include, without limitation, happy, relaxed, and bored. Illustrative examples of a negative type of emotion classifier can include, without limitation, sad, depressed, scared, frustrated, angry, nervous, or anxious.

Next, the alarming algorithm 1500 includes an operation 1506 of determining whether the one or more physiological variables exceed an upper or lower alarm limit. When the one or more physiological variables exceed an alarm limit (i.e., “Yes” at operation 1506), the alarming algorithm 1500 proceeds to an operation 1510 of triggering an alarm. When the one or more physiological variables do not exceed an alarm limit (i.e., “No” at operation 1506), the alarming algorithm 1500 proceeds to an operation 1508 of determining whether to trigger an alarm based on the type of emotion classifier determined in operation 1504.

In operation 1508, when the one or more physiological variables are in a normal range high side or normal range low side, and the emotion classifier determined from operation 1504 is classified as a negative type of emotion classifier (e.g., sad, depressed, scared, frustrated, angry, nervous, or anxious), the alarming algorithm 1500 proceeds to the operation 1510 of triggering the alarm. When the one or more physiological variables are in the normal range high side or the normal range low side, and the emotion classifier determined from operation 1504 is classified as a positive type of emotion classifier (e.g., happy, relaxed, or bored), the alarming algorithm 1500 does not trigger the alarm at operation 1512.

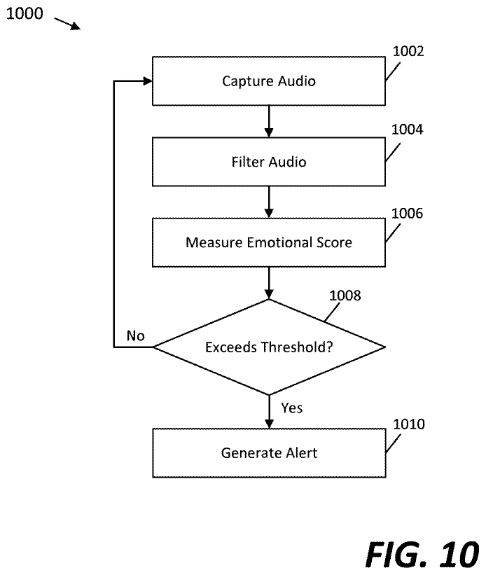

FIG. 10 illustrates another example of a method 1000 that can be performed by the communications system 20. The method 1000 includes an operation 1002 of capturing audio around the patient P using the room microphone and speaker unit 56. In some instances, the audio captured in operation 1002 can also be captured by the microphone and speaker unit 48, and/or by the microphone 1324 of the mobile device 22′. The audio captured in operation 1002 is captured regardless of whether the patient P is requesting assistance or not. Thus, the audio captured in operation 1002 is ambient noise that is continuously recorded and monitored.

Next, the method 1000 includes an operation 1004 of filtering the audio captured from operation 1002. The audio is filtered to identify audio that belongs to the patient P from audio detected from another patient in the same room as the patient P, from a caregiver providing care to the patient P, or from family members of the patient P who are visiting. The audio can be filtered by distinguishing speech from ambient noise, and analyzing one or more features of the detected speech such as the tone, pitch, jitter, energy, and the like to determine that the speech belongs to the patient P, and not someone else who is near the patient.

Next, the method 1000 includes an operation 1006 of measuring an emotional score from the filtered audio from operation 1004. Like in the examples described above, the emotional score can be measured by analyzing certain features in the speech of the patient P such as the tone, pitch, jitter, energy, rate, length and number of pauses, and the like. The emotional score measured in operation 1006 can indicate a likelihood that the patient P is going to harm himself or herself, or harm other patients and staff in the healthcare facility 10.

Next, the method 1000 includes an operation 1008 of determining whether the emotional score measured in operation 1006 exceeds a threshold, such that the patient P is at risk for harming himself or herself, or harming other patients and staff in the healthcare facility 10. When the emotional score does not exceed the threshold (i.e., “No” at operation 1008), the method 1000 returns to operation 1002 of capturing audio around the patient P.

When the emotional score does exceed the threshold (i.e., “Yes” at operation 1008), the method 1000 proceeds to operation 1010 of generating an alert. In instances when the emotional score indicates that the patient P is at risk for harming himself or herself, such as when the patient P is suicidal, the alert can be sent to a psychologist for immediate treatment to improve the emotional state of the patient P. In instances when the emotional score indicates that the patient P is at risk for harming other patients or staff in the healthcare facility, the alert can be sent to a security office or security guard to protect the other patients and staff from the patient P.

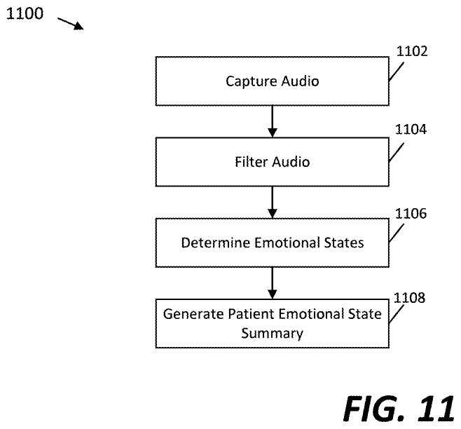

FIG. 11 illustrates another example of a method 1100 that can be performed by the communications system 20. The method 1100 includes an operation 1102 of capturing audio around the patient P, followed by an operation 1104 of filtering the audio captured from operation 1102 to identify audio that belongs to the patient P. Operations 1102, 1104 can be substantially similar to operations 1002, 1004 described above for the method 1000. For example, the audio can be captured in operation 1102 by the room microphone and speaker unit 56, and is captured regardless of whether the patient P is requesting assistance or not

Next, the method 1100 includes an operation 1106 of determining emotional states of the patient P based on the audio filtered in operation 1104. The emotional states can be determined in operation 1106 in accordance with the examples described above. For example, the emotional states can be determined by looking at features in the speech detected from the patient P, such as the tone, pitch, jitter, energy, rate, length and number of pauses, and the like.

Next, the method 1100 includes an operation 1108 of generating a patient emotional state summary, such as the one described above and shown in FIG. 9, based on the emotional states determined from operation 1106. Thus, the method 1100 is similar to the method 800, except instead of aggregating the emotion classifiers sent in the patient requests from the patient P, the method 1100 monitors the ambient noise around the patient P to continuously monitor the emotional states of the patent P while admitted in the healthcare facility 10.

The patient emotional state summary 906 that is generated in accordance with the operations of the method 1100 can be updated in real-time based on the audio received from the patient P, which allows the caregivers and staff in the healthcare facility 10 to view how patient P's stay in the healthcare facility 10 is going, and thereby improve the emotional state of the patient P. This information can be used by an office of patient experience, which tracks patient satisfaction across the healthcare facility 10 and handles issues with dissatisfied patients.

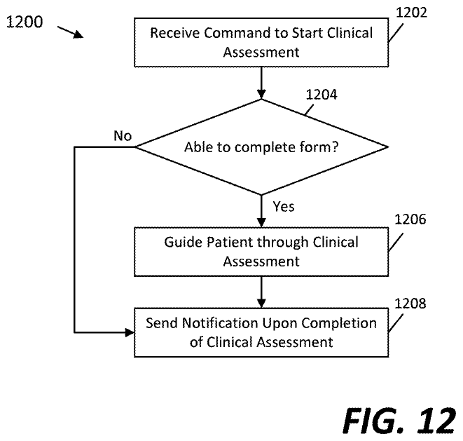

FIG. 12 illustrates an example of a method 1200 of assisting the patient P to complete written forms and clinical assessments with little or no intervention by the caregiver C. The method 1200 can be performed when the patient P has physical limitations that hinder or prevent them from writing with a pen or pencil such as due to advanced age, and when there is no family member or guardian to help them in completing the written forms and clinical assessments. The written forms and clinical assessments can be predefined by the healthcare facility 10. Examples of the types of written forms and clinical assessments that can be completed by the method 1200 can include, without limitation, depression screenings, demographic forms, family histories, delirium/confusion assessment method for the ICU (CAM-ICU), and the like.

In one embodiment, the method 1200 is performed by the workstation computer 62 when the clinical assessment module 210 is installed thereon, and using any of the microphone and speaker unit 48, room microphone and speaker unit 56, or mobile device 22′. In another embodiment, the method 1200 is performed by the patient bed 30 when the clinical assessment module 210 is installed thereon, and using the microphone and speaker unit 48 or the room microphone and speaker unit 56. In another embodiment, the method 1200 is performed by the mobile device 22′ when the clinical assessment module 210 is installed thereon.

As shown in FIG. 12, the method 1200 includes an operation 1202 of receiving a command from the clinician C to complete a form or clinical assessment. In some instances, the command is a voice command when the caregiver C is located proximate to the patient P, and the voice command is received by any of the microphone and speaker unit 48, room microphone and speaker unit 56, or mobile device 22′. The voice command can also alert the patient P that the form or clinical assessment will start such that the patient P can prepare and not be startled.

In some instances, operation 1202 includes verifying that the command is from an authorized caregiver, such as by detecting the presence of the caregiver C in the room with the patient P. The presence of the caregiver C can be detected by receiving a signal from a badge 66 worn by the caregiver C that indicates that the caregiver C is authorized to start the clinical assessment. Alternatively, the presence of the caregiver C can be detected by recognizing the voice of the caregiver such as by comparing characteristics of the caregiver's voice from the voice command to a known sample of the caregiver's voice. Once the caregiver C is verified, the caregiver C is free to perform other tasks with other patients in the healthcare facility 10.

Next, the method 1200 includes an operation 1204 of determining the patient P's capacity or ability to verbally complete the form or clinical assessment. For example, operation 1204 can include providing an introduction and building a rapport with the patient P by asking some initial questions (e.g., “How are you?”, “What is your name?”, etc.) before going through the form or clinical assessment. In some instances, operation 1204 can also determine whether the patient P is able to verbally interact such that the patient P is able to hear the questions and to verbally answer them. When non-responses or nonsensical responses are received from the patient P (i.e., “No” in operation 1204), the method 1200 proceeds to operation 1208 of sending a notification to the caregiver C that the patient P is unable to complete the clinical assessment.

When the capacity of the patient P is verified (i.e., “Yes” at operation 1204), the method 1200 proceeds to an operation 1206 of guiding the patient through one or more forms or clinical assessments and recording verbal responses from the patient P. The forms and clinical assessments include a plurality of audible queries to elicit verbal responses from the patient P.

Operation 1206 includes using natural language processing (NLP) to interact with the patient P in a human-like fashion for guiding the patient P to complete the forms and clinical assessments. For example, instead of providing a list of questions and recording answers, operation 1206 can include explaining the goal of the interaction/conversation with the patient P, returning the patient P's focus to the questions when the patient loses focus, providing examples when the patient P has difficulty in answering a question, and asking follow-up questions for clarification when ambiguous or nonsensical answers are received.

In some examples, operation 1206 includes recording the voice signatures of the patient P when the patient answers the questions to complete the form or clinical assessment. Also, operation 1206 can include communicating with the patient P in the patient P's native language (e.g., English, Spanish, French, Portuguese, Chinese, and the like).

Upon completion of the forms or clinical assessments, operation 1206 can include summarizing the conversation and thanking the patient for their time and patience. The answers recorded from the patient P in operation 1206 can be automatically stored in the electronic medical record (EMR) or electronic health record (EHR) of the patient on EMR server 64.

Next, the method 1200 includes an operation 1208 of sending a notification to the caregiver C when the clinical assessment is complete. For example, the notification can be sent to the mobile device 22 of the caregiver C to alert them that the form or clinical assessment has been completed. Advantageously, the caregiver C can be notified about the completion of the form or clinical assessment when the caregiver C is located in a different area than the patient P in the healthcare facility 10 such as when the caregiver C is another patient room helping another patient. Also, the notification can be sent to the workstation computer 62, which as described above, can be located in a nurses' station where the caregiver C works when not working directly with the patient P, such as where the caregiver C performs administrative tasks.

FIG. 13 schematically illustrates an example of a mobile device 22, 22′ which can be used to implement aspects of the communications application 200, described above. The mobile device 22, 22′ includes a processing unit 1302, a system memory 1308, and a system bus 1320 that couples the system memory 1308 to the processing unit 1302. The processing unit 1302 is an example of a processing device such as a central processing unit (CPU).

The system memory 1308 includes a random-access memory (“RAM”) 1310 and a read-only memory (“ROM”) 1312. The ROM 1312 can store input/output logic containing routines to transfer information between elements within the mobile device 22, 22′.

The mobile device 22, 22′ can also include a mass storage device 1314 that is able to store software instructions and data. The mass storage device 1314 is connected to the processing unit 1302 through a mass storage controller connected to the system bus 1320. The mass storage device 1314 and its associated computer-readable data storage media provide non-volatile, non-transitory storage for the mobile device 22, 22′.

Although the description of computer-readable data storage media contained herein refers to a mass storage device, it should be appreciated by those skilled in the art that computer-readable data storage media can be any available non-transitory, physical device or article of manufacture from which the device can read data and/or instructions. In certain embodiments, the computer-readable storage media comprises entirely non-transitory media. The mass storage device 1314 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The mobile device 22, 22′ operates in a networked environment using logical connections to devices through the network 100. The mobile device 22, 22′ connects to the network 100 through a network interface unit 1304 connected to the system bus 1320. The network interface unit 1304 may also be utilized to connect to other types of communications networks and devices, including through Bluetooth and Wi-Fi.

The mobile device 22, 22′ can also include an input/output controller 1306 for receiving and processing inputs and outputs from a number of input devices. Examples of input devices may include, without limitation, a touchscreen display device and camera.

The mobile device 22, 22′ further includes a speaker 1322, and a microphone 1324, which can be used to record the audio and speech input from the patient P. The mobile device 22, 22′ can transfer the recorded audio and speech input from the patient P to the nurse call server 60 or WAM 26 using the connection to the network 100 through the network interface unit 1304.

The mass storage device 1314 and the RAM 1310 can store software instructions and data. The software instructions can include an operating system 1318 suitable for controlling the operation of the mobile device 22, 22′. The mass storage device 1314 and/or the RAM 1310 also store one or more software applications 1316, that when executed by the processing unit 1302, cause the device to provide the functionality of the mobile device 22, 22′ discussed herein. The software applications 1316 can include the communications application 200, as described above.

The various embodiments described above are provided by way of illustration only and should not be construed to be limiting in any way. Various modifications can be made to the embodiments described above without departing from the true spirit and scope of the disclosure.

What is claimed is:

1. A communications system for a healthcare facility comprising:
at least one processing device; and
a memory device storing instructions which, when executed by the at least one processing device, cause the at least one processing device to:
receive speech from a patient;
convert the speech to text to determine a patient request;
process the speech to determine an emotion classifier;
generate a message containing the patient request and the emotion classifier;
identify a caregiver based on the patient request; and
send the message to the caregiver.

2. The communications system of claim 1, wherein the instructions further cause the at least one processing device to:
determine an urgency level from the speech; and
include the urgency level in the message.

3. The communications system of claim 1, wherein the instructions further cause the at least one processing device to:
generate the message for display on a mobile device of the caregiver.

4. The communications system of claim 1, wherein the instructions further cause the at least one processing device to:
map the text to a predefined care category; and
match the predefined care category to a role of the caregiver.

5. The communications system of claim 1, wherein the instructions further cause the at least one processing device to:
aggregate emotion classifiers of the patient; and
generate a patient emotional state summary from the aggregate of the emotion classifiers.

6. The communications system of claim 1, wherein the instructions further cause the at least one processing device to:
capture audio from an area where the patient is located; and
generate a patient emotional state summary based on the audio.

7. The communications system of claim 6, wherein the instructions further cause the at least one processing device to:
determine an emotional score based on the audio; and
generate an alert when the emotional score indicates that the patient is at risk for harming themself or others.

8. The communications system of claim 1, wherein the instructions further cause the at least one processing device to:
receive a voice command from the caregiver to start a clinical assessment, the clinical assessment including a plurality of audible queries eliciting verbal responses from the patient;
use natural language processing to guide the patient through the clinical assessment;
record the verbal responses from the patient; and
send a notification to the caregiver upon completion of the clinical assessment.

9. A method of healthcare based communications comprising:
receiving speech from a patient;
converting the speech to text to determine a patient request;
processing the speech to determine an emotion classifier;
generating a message containing the patient request and the emotion classifier;
identifying a caregiver based on the patient request; and
sending the message to the caregiver.

10. The method of claim 9, further comprising:
determining an urgency level from the speech; and
including the urgency level in the message.

11. The method of claim 9, further comprising:
generating the message for display on a mobile device of the caregiver.

12. The method of claim 9, further comprising:
mapping the text to a predefined care category; and
matching the predefined care category to a role of the caregiver.

13. The method of claim 9, further comprising:
aggregating emotion classifiers of the patient; and
generating a patient emotional state summary from the aggregating of the emotion classifiers.

14. The method of claim 9, further comprising:
capturing audio from an area where the patient is located; and
generating a patient emotional state summary based on the audio.

15. The method of claim 14, further comprising:
determining an emotional score based on the audio; and
generating an alert based on the emotional score when the emotional score indicates that the patient is at risk for harming themself or others.

16. The method of claim 9, further comprising:
receiving a voice command from the caregiver to start a clinical assessment, the clinical assessment including a plurality of audible queries;
using natural language processing to guide the patient through the clinical assessment;
recording verbal responses from the patient; and
sending a notification to the caregiver upon completion of the clinical assessment.

17. A non-transitory computer readable storage medium storing instructions, which when executed by at least one processing device, cause the at least one processing device to:
receive speech from a patient;
convert the speech to text to determine a patient request;
process the speech to determine an emotion classifier;
generate a message containing the patient request and the emotion classifier;
identify a caregiver based on the patient request; and
send the message to the caregiver.

18. The non-transitory computer readable storage medium of claim 17, comprising further computer readable instructions that cause the at least one processing device to:
determine an urgency level from the speech; and
include the urgency level in the message.

19. The non-transitory computer readable storage medium of claim 17, comprising further computer readable instructions that cause the at least one processing device to:
generate the message for display on a mobile device of the caregiver.

20. The non-transitory computer readable storage medium of claim 17, comprising further computer readable instructions that cause the at least one processing device to:
map the text to a predefined care category; and
match the predefined care category to a role of the caregiver.
